(19) United States
(12) Patent Application Publication
(10) Pub. No.: US 2021/0193258 A1
(43) Pub. Date: Jun. 24, 2021

(54) DETECTION OF CHANGES IN GENE EXPRESSION ATTRIBUTABLE TO CHANGES IN CELL MORPHOLOGY

(71) Applicants: Jeffrey Hall, Norwalk, CT (US); Richard James, Norwalk, CT (US)

(72) Inventors: Jeffrey Hall, Norwalk, CT (US); Richard James, Norwalk, CT (US)

(21) Appl. No.: 16/922,494

(22) Filed: Jul. 7, 2020

Related U.S. Application Data
(60) Provisional application No. 62/871,590, filed on Jul. 8, 2019.

(51) Int. Cl. G16B 25/10; G16B 5/20

(52) U.S. Cl. CPC G16B 25/10; G16B 5/20

(57) ABSTRACT

Methods and compositions to detect morphological impact on gene expression from gene expression signals. Locations of marginally-expressed probesets are measured relative to the location of expressed and non-expressed probesets. A set of scores are generated, which may be used to detect effects of cell morphology on the mechanism of gene expression; for example, the effect of organism age, or the state of mitochondrial function, or the impact of CRISPR editing, or membership in sub-populations within clinical trials for whom treatment is safe and/or effective.

### FIELD OF THE INVENTION

The present invention relates to laboratory or in silico detection of apparent changes in the mechanism of gene expression. The invention includes methods and compositions for detecting apparent changes in the mechanism of gene expression that are due to changes in cell morphology, especially as they relate to the natural or pathological aging process of tissue in an organism.

### BACKGROUND OF THE INVENTION

Since gene expression is influenced by the shape of the space under which transcription occurs, changes in gene expression can arise out of changes in cell morphology. Such changes may be caused, for example, by alterations in the number and integrity of the mitochondria which are responsible for maintaining the positional structure of everything within the cell. But other factors such as the size of the nucleolus, the integrity of nuclear lamins, CRISPR editing, and the arrangement of chromatin can also induce changes in cell morphology. Altogether, the factors associated with changes in cell morphology contribute to, and in turn can be the result of, the processes of cellular aging, oncogenesis or other cellular pathologies.

The ability to detect these morphology-induced changes would provide, in one aspect, an inexpensive and one-off measure of “biological age” of the organism which could be performed on any type of tissue. The ability to detect changes to gene expression due to cell morphology would also, in another aspect, provide a way to measure the progress of disease states that specifically impact cell morphology.

### SUMMARY OF THE INVENTION

It is our object to detect apparent changes in gene expression due to cell morphology, given a measure of gene expression likelihood that is consistent across chromosomes of a sample, or at least across chromosome arms.

To detect apparent changes in gene expression due to changes in cell morphology, we partition the genes whose expression is measured in the input signal into four classes:
1. Genes that are assumed to be, or known with high probability to be, expressed;
2. Genes that are only suspected to be expressed;
3. Genes that are assumed NOT to be, or known with high probability NOT to be, expressed;
4. Genes that do not fall into any of the previous three classes.

If the input signal consists, for example, of the output of an oligonucleotide array, then classes 1 and 2 may be taken, in one embodiment, to be those genes found to have “P” and “M” calls from Wilcoxon sign-rank tests on the array probes. In this case, class 3 can be chosen, for example, to be genes hybridizing with “A” (Absent) probesets with some high p-value, say ≥0.5. Properly speaking, probesets with high p-values from the Wilcoxon sign-rank test are not identified as absent, but rather are identified as probesets whose null hypothesis that the probeset does not correspond to an expressed gene is not rejected. The logical Law of the Excluded middle does not hold here; so that, in many practical embodiments of our invention, there are thousands of class 4 genes. Other embodiments can use genomic information about the sample's actual tissue type as follows: if a gene is known or assumed to never be expressed or mis-expressed in a given sample's tissue type, then it may be assumed to always be in class 3.

Consider classes as they appear on a single chromosome. The genes from each of the classes 1, 2, and 3 on a single chromosome could be represented as an ordered sequence of DNA locations on that chromosome. For every chromosome arm, each of these sequences may be thought of as a discrete probability distribution on the possible locations (or ranks) on that chromosome arm. Call these sequences of gene positions or ranks S_{1,X}, S_{2,X} and S_{3,X}, where X is a Chromosome Arm (see glossary below.)

Two key insights here are (1) that the sequences S_{1,X}, S_{2,X} and S_{3,X} are samples drawn from some unspecified random variable, and (2) that given any measure of correlation m between the sampled distributions, mis-expression due to changing gene morphology would be expected to contribute to both m(S_{2,X}, S_{1,X}) and m(S_{2,X}, S_{3,X}) monotonically, in opposite directions.

In one aspect, this embodiment generates the scores m₁₂=m(S_{2,X}, S_{1,X}) and m₂₃=m(S_{2,X}, S_{3,X}) as markers of the behavior of gene expression regulation associated with changes in cell morphology. In another aspect, these scores are used with some desired objective measure associated with the samples from which the scores were generated two for each chromosome or arm—to interpret the scores. The interpretation of these markers may be calibrated, as we will exemplify in the disclosure below, with weak but easy to measure markers of gene expression health. In one embodiment, we use chronological age as just such an objective measure.

### Advantages of the Invention

The present embodiment provides methods and compositions to detect the degree of morphological impact on gene expression from a gene expression signal, such as from standard RNA microarray or RNAseq signals. Our invention uses marginally-expressed probesets—which are commonly discarded by other methods of gene expression analysis—to measure the state of cell morphology.

Our invention provides a means, in one aspect, to predict the chronological age of an individual from a single tissue sample. Our invention is applicable across a variety of different tissue types and can also be used to detect the relative rate of aging of a particular tissue from a single individual compared to other tissue from the same individual.

Since any biomarker obtained through our invention indirectly measures the efficiency of the cell's mitochondria in maintaining the internal structure of the cell, our invention can also be used to detect mitochondrial health in situations where other markers of mitochondrial health are impractical; for example, in retrospective population, pharmacometric, or gene-editing studies; or in the study and treatment of Alzheimer's disease.

Still other objects and advantages of the invention will in part be obvious to those skilled in the art, and will in part be apparent from the specification and drawings.

### BRIEF DESCRIPTION OF THE DRAWINGS

FIG. 1 shows an exemplary arrangement of an embodiment of our invention. FIG. 2 shows an exemplary arrangement of another embodiment of our invention. FIGS. 3, 4, 5 and 6 show histograms of the scores generated by a prototype of our invention for both arms of a chromosome from a Homo s. kidney biopsy.

FIG. 1 is a simplified flow diagram illustrating the generation of Comparative Expression Scores from RNA microarray data.

FIG. 2 is a simplified flow diagram illustrating the calibration of model parameters to an objective measure from Comparative Expression Scores.

FIG. 3 is a histogram of m₁₂ Comparative Expression Scores for the long arms of chromosomes from a Homo s. kidney biopsy.

FIG. 4 is a histogram of m₁₂ Comparative Expression Scores for the short arms of chromosomes from a Homo s. kidney biopsy.

FIG. 5 is a histogram of m₂₃ Comparative Expression Scores for the long arms of chromosomes from a Homo s. kidney biopsy.

FIG. 6 is a histogram of m₂₃ Comparative Expression Scores for the short arms of chromosomes from a Homo s. kidney biopsy.

### REFERENCE NUMERALS

101 Gene Expression Signal Data
102 Annotation Data for Assay
103 Process to Normalize Gene Expression Data
104 Normalized Gene Expression Table
105 Process to Estimate Likelihood of Gene Expression
106 Expression Likelihood Table
107 Process to Classify Genes
108 Classified Gene Expression Table
109 Process to Assign Genes to Arms and Locations
110 Chromosome Maps
111 Process to Generate Comparative Expression Scores
112 Array of Comparative Expression Scores
201 Table of Comparative Expression Scores
202 Table of Objective Measures
203 Iteration Subprocess Entrance
204 Process to Fit Model
205 Score P-Value Table
206 Provisional Set of Model Parameters
207 Process to Select Predictive Arms
208 Iteration Subprocess Condition
209 Process to Extract a Subset of Comparative Expression Scores
210 Subset of Comparative Expression Scores
211 Accepted Set of Model Parameters
212 Model

### DETAILED DESCRIPTION OF THE PREFERRED EMBODIMENT

### Glossary of Key Terms

Assay Annotation Data—An empty or nonempty table that lists, for Probesets in a Gene Expression Detection Method, the chromosome that the Probeset is assumed to lie on, the arm (if applicable) that the Probeset is assumed to lie on, the Gene (if any) that the Probeset is assumed to be a part of, and the location, if known, that the Probeset is assumed to have on that chromosome or chromosome arm.
   Alternately, the Assay Annotation Data may be a table that lists, for all of the Probesets in a Gene Expression Detection Method, the Gene (if any) that the Probeset is assumed to be a part of, and the corresponding information (chromosome, arm, location) for each Gene that the Probeset is assumed to be a part of. Other obvious variations (e.g., a subset or superset of the Genes with known Probesets, or data with Gene chromosomes but no Probeset locations, etc.) are possible.
   The contents of Assay Annotation Data are a signal that may be transmitted to an embodiment of the invention via a file, or a text string (if an embodiment of the invention is implemented as software), or via any other method. If the means to normalize the gene expression values calculates or imputes any of these values itself, then the table need not contain those values. In particular, in some embodiments, the Assay Annotation Data is empty.
Autocorrelation—A function that, given a sequence, returns a measure of the correlation of a sequence with itself.
Biological Age—When an individual of a species can be placed in a cohort of individuals with similar physiological properties or other measured or imputed properties, the average age of that cohort is called the Biological Age of that individual.
Chromosome Arm—An arm of a chromosome with a centromere. When referring to chromosomes with no centromere, or whose Genes all lie on one arm, we also refer to that whole chromosome as a Chromosome Arm.
Chromosome Location—Any assignment of one or more ordinal numbers to the location of a Gene (or of a Probeset, or pseudogene, or any other chosen set of DNA sequences) on a chromosome, so that the order of the DNA sequence locations of Genes (or Probesets, or pseudogene, etc.) on each Chromosome Arm is preserved.
Chromosome Map—A list of Chromosome Locations, all of which lie on the same Chromosome Arm. The contents of a Chromosome Map are a signal that may be transmitted to an embodiment of the invention via a file, or a text string (if an embodiment of the invention is implemented as software), or via any other method.
Chronological Age—The age of an individual, measured in some unit of time (years, days, etc.)
Cell Morphology—The structure, shape and appearance of a cell, especially as it relates to the internal structure of a cell.
Gene—A set of DNA sequence locations on a chromosome which, when or if it is expressed, is assumed to be expressed together.
Gene Expression Detection Method—A method to measure RNA levels in the tissue, using a microarray, RNASeq, or other commercially available or custom protocol.
Gene Expression Table—A table that lists, for each sample that has been processed by a Gene Expression Detection Method, and for each Probeset that is detected by that Gene Expression Detection Method, an ordinal measure of detection (for example, a measure of the amount detected, or the probability of detection, or any other ordinal measure.)
   The contents of a Gene Expression Table are a signal that may be transmitted to an embodiment of the invention via a file, or a text string (if an embodiment of the invention is implemented as software), or via any other method.
Imputation—See Prediction.
Likelihood—A measure of confidence that a hypothesis is true; for example, the calculated p-value of an event, or the rank of an event with respect to its sample space.
Measure Of Similarity—A measure of how much two samples, two random variables, or two sequences are alike.
Model—An assumed but imperfectly known relation between a set of random variables.
   If the assumed relation has a given mathematical form, then the Model may be described as a string of a well-known form which we don't define precisely here (see any introductory Statistics or Mathematical Modeling textbook.) For example:

V₁˜V₂+V₃V₄.
describes a Model where the random variable V₁ is assumed to equal some constant times V₂ plus some other constant times V₃ times V₄, with an error term not specified.
The contents of a Model are a signal that may be transmitted to an embodiment of the invention via a file, or a text string (if an embodiment of the invention is implemented as software), or via any other method.
Model Fit—Given a Model and a table of simultaneously observed values of the random variables in the Model, a Model Fit (or fitted model) is an estimate for the parameters (the constants in the above definition) in the Model, assuming the table of simultaneously observed values was a representative sample.
   We note that if the Model was a linear model (in particular, if the random variables are Gaussian) then any device or process that implements the method Linear Regression provides such a fit, and that for much more general models with arbitrary random variables, the celebrated Metropolis-Hastings algorithm provides such a fit, if enough data is available.
Normalization—Given a Gene Expression Table, a Normalization method is a process that transforms the Gene Expression Table into a table of comparable expression values of Genes.
Partition—A division of a set S into disjoint subsets, whose union is S.
Prediction—Given a Model of the form Y ˜something, and a fit of that Model, then substituting the fitted parameters and observed values of the random variables on the right-hand side of the model string yields a number, which is called the predicted or imputed value of Y for that observation. This process is called Prediction or Imputation.
Probability Distribution—For a “sample space” or “sample set” S, a mapping P from some of the subsets S to the interval {0≤P(T)≤T⊆S}, so that:
   1. P(S)=1 and P(∅)=0;
   2. P(T₁∪T₂)≤P(T₁)+P(T₂) and P(T₁∩T₂)≤min(P(T₁), P(T₂)); and
   3. if T₁∩T₂=∅, then P(T₁∪T₂)=P(T₁)+P(T₂)
Probeset—Either: (1) a Probeset of an oligonucleotide array, or (2) a unique RNA sequence found by RNASeq or other technologies, or (3) a protein or RNA sequence fragment or set of fragments detected, considered as a sequence of amino acids or oligonucleotides. In any case, a Probeset's presence or absence is a thing detected in a tissue sample by a Gene Expression Detection Method, creating a Gene Expression Table.
   Note that, in the first case, a Probeset corresponds to a set of DNA sequence locations, and in the second case, a unique RNA sequence locations. Note also that, in the prior literature, “probeset” is used in the first case; the generalization defined here is intended to simplify the description of the present embodiment.
Rank—If S is an ordered set, then the Rank of s∈S is x if and only if
   1. 1≤x≤|S|, where |T| refers to the size of set T;
   2. x is a positive integer or x is one-half of a positive integer;
   3. a positive integer n is less than x if n<|{t∈S|t<s}|, and;
   4. a positive integer n is greater than x if n>|{t∈S|t<s}|.
   Likewise, if S is a sequence rather than a set, the Ranks of S, written rank(S), is the sequence of Ranks for each entry in S. For example, the Ranks of [1.0, 2.0, 4.1, 6.5, −4.3] are [3, 2, 4, 5, 1], and the Ranks of [3.2, 2.0, 2.0, 1.0] are [3, 2.5, 2.5, 1]
Sequence—An ordered list of arbitrary items, which may be repeated. For example, [1, 2, 2, 2, 4, 1, 5] and [‘a’,‘a’,‘a’,‘a’,‘a’,‘a’,‘a’] are both sequences with 7 entries.
Table Of Objective Measures—A table that lists, for a sample that has been processed by a Gene Expression Detection Method, the value of some objective function, or the observed, estimated or assumed value of some objective function or property of that sample. For example, the Chronological Ages of individuals when they were sampled, the mean number of mitochondria per cell observed for each sample, etc.
   The contents of a Table Of Objective Measures are a signal that may be transmitted to an embodiment of the invention via a file, or a text string (if an embodiment of the invention is implemented as software), or via any other method.

### DETAILED DESCRIPTION

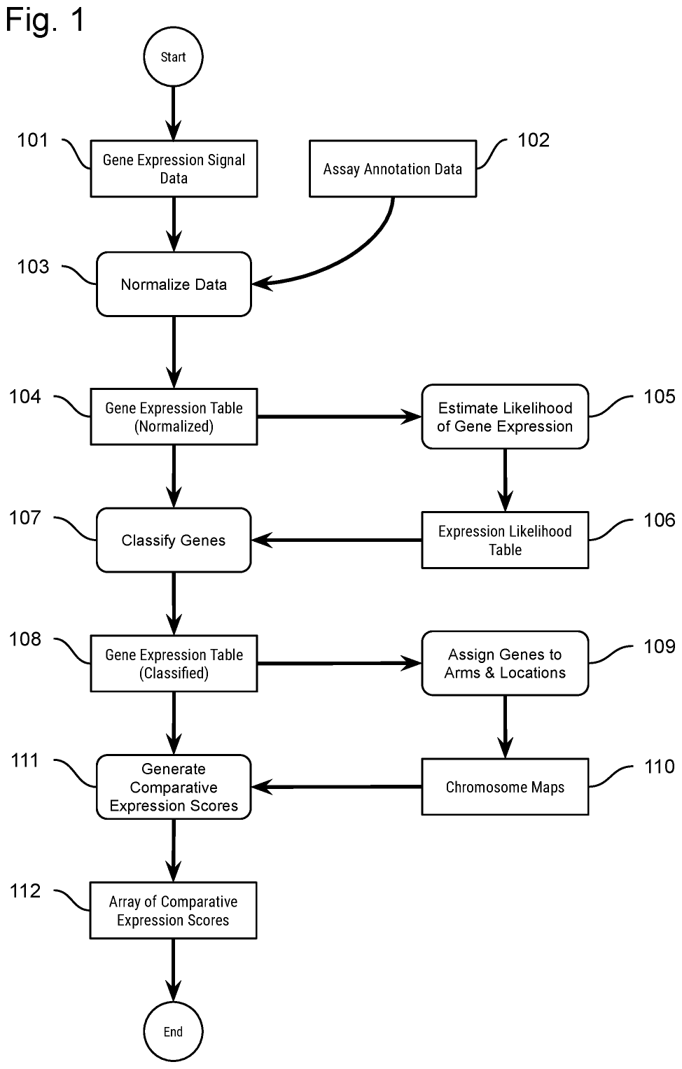

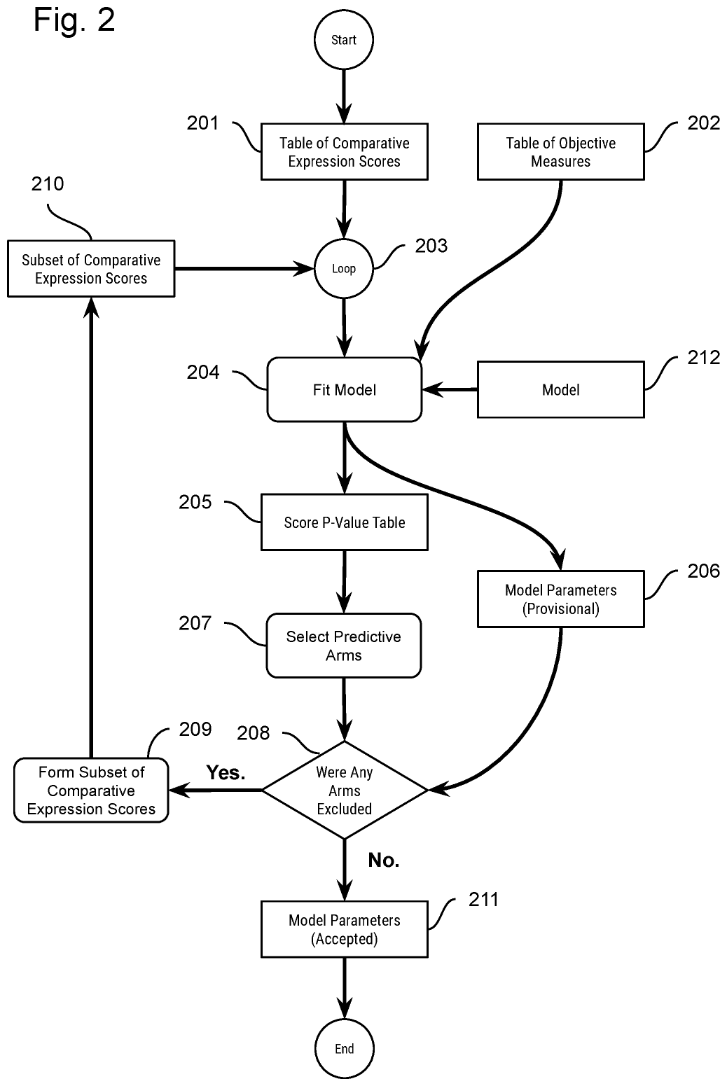

The flow diagrams in FIG. 1 and FIG. 2 show an exemplary arrangement of a preferred embodiment of our invention. The preferred embodiment in one aspect provides a system to process a raw Gene expression signal from a single tissue sample of an organism into a description of apparent previous changes in Gene expression that are ascribed to changes in Cell Morphology of that organism (FIG. 1). In another aspect, the preferred embodiment provides a method (FIG. 2) to calibrate these scores to predict an objective measure, which in one embodiment is Chronological Age.

The present embodiment is a system that receives Gene Expression Signal Data 101 from plant, animal or other eukaryote tissue. A measure of RNA expression levels for Probesets in the tissue is found, for example by a Gene Expression Detection Method. As FIG. 1 shows, the expression data 101 presented to the system described in this application is normalized, 103, so that expression measures are comparable across Genes of the sample. If the normalization process does not calculate or assume a correspondence between Genes, Probesets, and Chromosome Locations, then the normalization process uses the Assay Annotation Data 102 to detect these values. In either case, the normalization process 103 produces a normalized Gene Expression Table 104.

From the normalized Gene Expression Table 104, we estimate, 105, the Likelihood that each Probeset in each sample is expressed. For example, if raw probeset data is available, then the MAS5 algorithm may be used to estimate these Likelihoods; or RNAseq p-values may be used; or if multiple samples (treatment/control, time series, etc.) are available, then any measure of Likelihood of up/down regulation may be used. In our preferred embodiment, the result is a table 106 of Likelihoods for the hypothesis that a Gene is not expressed, for each Gene in the (normalized) Gene Expression Table 102 for which good data was obtained from the normalization process 103 above.

Now for the sample, the Genes for which data is available are classified 107 into classes. In our preferred embodiment, we partition into four classes: Class 1 are Genes with are found with a high probability to be expressed (e.g., Genes where the probability that the Gene is not expressed is p<0.001, or some other cutoff.) Class 2 are Genes that are reasonably suspected to be expressed (e.g., Genes where 0.002<p≤0.005, or some other pair of bounds.) Class 3 are Genes that are assumed not to be expressed (e.g., Genes where p>0.5, or some other bound.) Class 4 consists of all Genes that are not in classes 1, 2, or 3. We now have a table 108 of four classes of Genes for the sample.

Each of the Probesets is assumed to exist on a known Chromosome Arm of the sample organism's genome. Using a standard sequenced genome for the sampled organism's species, or by some other method, such as: by modeling; or by actually sequencing the genome of the individual whose Gene expression signal 101 is being examined; or by assuming that unique Probesets correspond to unique Genes; or by using any operator-defined assignment of Probesets to Genes; or by using any operator-defined assignment of Probesets to Chromosome Locations; we assign 109 to each Gene in the classified Gene Expression Table 108 a unique chromosome and arm, and a unique position on that Chromosome Arm, creating a Chromosome Map 110 for each Chromosome Arm. In an alternative embodiment, each Chromosome Map may be generated from any ranking of the ordered Genes that are known to lie on that Chromosome Arm, thought of as a polymer sequence, rather than the actual Chromosome Locations. Those who are skilled in the art who examine this process will readily see other technically equivalent ways to generate Chromosome Maps.

Now given any measure n of similarity or correlation between Gene Ranks or

Chromosome Locations, we generate 111 the values

m₁₂=m(S_{2,X},S_{1,X})

and

m₂₃=m(S_{2,X},S_{3,X})

for each Chromosome Arm X of the sample.

We call these resulting numbers the Comparative Expression Scores.

In our preferred embodiment, we choose a natural choice of m: we think of each S_{i,X} as a random sample from some unspecified random variable on the same underlying sample space, and after calculating an Autocorrelation of each sequence S_{i,X}, define mᵢⱼ=m(S_{i,X}, S_{j,X}) to be the modified Whitney-Mann Wilcoxon statistic between S_{i,X} and S_{j,X} for (i,j)∈{(1,2), (2,3)}.

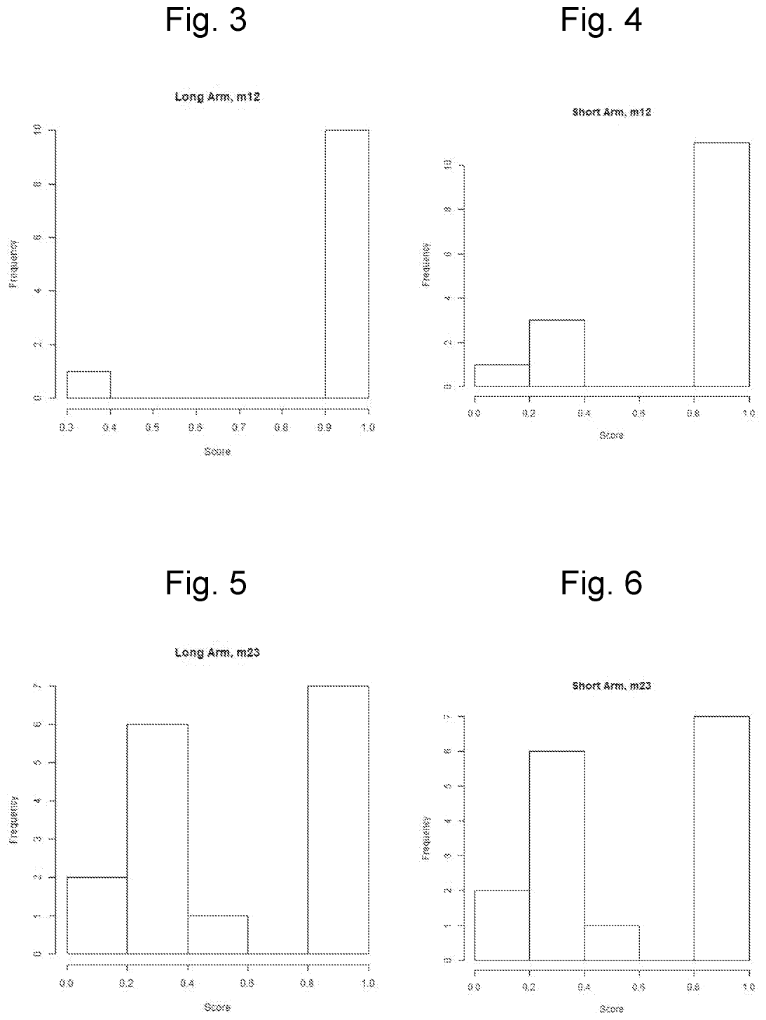

The result is an array 112 of m12 and m23 values, 2*N for each sample, where N is the number of chromosomes used, for each chromosome and arm of each sample. FIG. 3 shows the distributions and values for a particular Chromosome Arm for tissue from a Homo s. kidney biopsy.

The array of Comparative Expression Scores 112 is fine-grained information that captures how the mechanism of Gene expression of the sampled organism was affected by Cell Morphology.

FIG. 2 provides an exemplary way to interpret the Comparative Expression Scores for a particular cell type and tissue type of a given species. Arrays of Comparative Expression Scores 112 from two or more samples are joined to form a table of Comparative Expression Scores 201 for this system.

Next we fit 204 any Model 212 that relates a table of values of an objective function 202 that is assumed to be weakly correlated with efficiency of Gene expression to the Comparative Expression Scores 201. In one embodiment, this objective function is the Chronological Age of the individual providing each sample and this Model is chosen by the operator to be physiologically reasonable. For example, a reasonable Model for age might be:

Age˜M₁₂+M₂₃

where the scores m_{i,j}=m(S_{i,X},S_{j,X}) 201 are observed values of the random variable Mᵢⱼ. In alternative embodiments, the objective function may be a physiological variable, a drug or treatment response, the presence or count of adverse events, the presence of a diagnosis, the observed effect of CRISPR or other Gene editing technique, or any other observed or imputed clinical value; and the Model is likewise chosen by the operator to be physiologically reasonable.

The result of fitting the Model 204 to the objective function values 202 and Comparative Expression Scores 201 is a table of provisional Model Parameters 206 together with a table of p-values 205 or other form of Likelihood for each of the Comparative Expression Scores. If there are Chromosome Arms whose scores have high p-values, they may be excluded at the operator's discretion, selecting only the predictive Chromosome Arms, 207. If any Chromosome Arms were thus excluded, 208, the corresponding subset of Comparative Expression Scores for that Chromosome Arm is also excluded, 209, forming a new table 210 of Comparative Expression Scores for the included Chromosome Arms, and the process is repeated, 203. Otherwise, at 208, the process stops with an accepted set of model parameters 211. These parameters and the Model provide a method for imputing the objective function from the Comparative Expression Scores.

It will thus be seen that the objects set forth above, among those made apparent from the preceding description, are efficiently attained and, because certain changes may be made in carrying out the above method and in the construction(s) set forth without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described and all statements of their scope of the invention which, as a matter of language, might be said to fall therebetween.

What is claimed:

1. A system, comprising:
Gene expression signal data from an organism;
Assay Annotation Data;
a first means to normalize the Gene expression signal values;
a normalized Gene Expression Table generated by first means;
a second means to generate Gene expression Likelihoods from a normalized Gene Expression Table;
a table of expression Likelihoods generated by second means;
a third means to partition the Genes based on expression Likelihoods, into three or more classes;
a classified Gene Expression Table generated by third means;
a fourth means to generate a Chromosome Map for one or more Chromosome Arms from the classified Gene Expression Table;
a set of Chromosome Maps for each Chromosome Arm generated by fourth means;
a fifth means to generate a measure of similarity between any two given classes of Genes in the same Chromosome Map;
an array of those generated measures of similarity.

2. The system of claim 1, further comprising:
a Table of Comparative Expression Scores for multiple samples;
a Table Of Objective Measures;
a first Model relating Comparative Expression Scores to the random variable from which the data in the Table Objective Measures is assumed to be drawn;
a sixth means to fit first Model with a table of Comparative Expression Scores;
a fit of first Model against the Table Of Objective Measures, resulting from sixth means, comprising:
i) an estimate of the model parameters; and
ii) an estimate of the predictive p-value for each Comparative Expression Scores for each Chromosome Arm from that Model Fit;
a decision to exclude non-predictive Chromosome Arms;
a table, the Subset of Comparative Expression Scores, consisting of scores from Chromosome Arms that were not excluded; and
a table of Model Parameters (Accepted), comprising:
i) an estimate of the model parameters for the accepted Chromosome Arms; and
ii) an estimate of the predictive Likelihood for each Chromosome Arm from that Model Fit for the accepted Chromosome Arms.

3. The system of claim 2 used to detect the Biological Age of the individual sampled wherein the Table Of Objective Measures for the samples are known or estimated Chronological Age of the individual organisms sampled.

4. The system of claim 2 used to detect the mitochondrial behavior or physiology in the individual sampled wherein the Table Of Objective Measures for the samples are known or estimated measures of mitochondrial function of the individual sampled, for example the average number of mitochondria per cell, the average number of healthy mitochondria per cell, or the presence, exclusion or absence of diagnoses related to mitochondrial function.

5. The system of claim 1 used to allow the operator to detect the effect of CRISPR or other gene-editing technique on the Gene expression of the tissue that was sampled, further comprising:
a display or report of the generated Comparative Expression Scores.

6. The system of claim 2 used to detect, given a set of samples with known responses to a drug, a surgical treatment, chemical exposure or any other stimuli, intervention or exposure, or combination of drugs, surgical treatments, exposures, etc., sub-populations of the individuals represented by the samples who are susceptible or non-susceptible to the drug, stimuli, or treatment, based on their Comparative Expression Scores and the known responses.

7. A method, comprising:
accessing Gene expression signal data from an organism;
accessing Assay Annotation Data;
normalizing the Gene expression signal values;
generating Gene expression Likelihoods from a normalized Gene Expression Table;
partitioning the Genes based on expression Likelihoods, into three or more classes;
generating a Chromosome Map for one or more Chromosome Arms from the classified Gene Expression Table;
generating a measure of similarity between any two given classes of Genes in the same Chromosome Map.

8. The method of claim 7, further comprising:
accessing a Table of Comparative Expression Scores for multiple samples;
accessing a Table Of Objective Measures;
initializing a second Model relating Comparative Expression Scores to the random variable from which the data in the Table Objective Measures is assumed to be drawn;
fitting second Model with a table of Comparative Expression Scores, com-promising:
i) generating an estimate of the model parameters; and
ii) generating an estimate of the predictive p-value for each Comparative Expression Scores for each Chromosome Arm from second Model Fit;
deciding to exclude non-predictive Chromosome Arms;
generating a table, the Subset of Comparative Expression Scores, consisting of scores from Chromosome Arms that were not excluded; and
generating a table of Model Parameters (Accepted), comprising:
i) generating an estimate of the model parameters for the accepted Chromosome Arms; and
ii) generating an estimate of the predictive Likelihood for each Chromosome Arm from second Model Fit for the accepted Chromosome Arms.

9. The method of claim 9 used to detect the Biological Age of the individual sampled wherein the Table Of Objective Measures for the samples are known or estimated Chronological Age of the individual organisms sampled.

10. The method of claim 8 used to detect the mitochondrial behavior or physiology in the individual sampled wherein the Table Of Objective Measures for the samples are known or estimated measures of mitochondrial function of the individual sampled, for example the average number of mitochondria per cell, the average number of healthy mitochondria per cell, or the presence, exclusion or absence of diagnoses related to mitochondrial function.

11. The method of claim 7 used to allow the operator to detect the effect of CRISPR or other gene-editing technique on the Gene expression of the tissue that was sampled, further comprising:
a display or report of the generated Comparative Expression Scores.

12. The method of claim 8 used to detect, given a set of samples with known responses to a drug, a surgical treatment, chemical exposure or any other stimuli, intervention or exposure, or combination of drugs, surgical treatments, exposures, etc., sub-populations of the individuals represented by the samples who are susceptible or non-susceptible to the drug, stimuli, or treatment, based on their Comparative Expression Scores and the known responses.
